(19) United States
(12) Patent Application Publication
(10) Pub. No.: US 2009/0325148 A1
(43) Pub. Date: Dec. 31, 2009

(54) Bead Array Reader Based-Hemagglutination and Hemagglutination Inhibition Assay

(75) Inventors: Anatoly KACHURIN, Orlando, FL (US); Vaughan WITTMAN, Oviedo, FL (US); Olga KACHURINA, Orlando, FL (US); Tenekua TAPIA, Orlando, FL (US)

Correspondence Address: MERCHANT & GOULD PC, P.O. BOX 2903, MINNEAPOLIS, MN, 55402-0903, US

(73) Assignee: VAXDESIGN INC.

(21) Appl. No.: 12/494,741

(22) Filed: Jun. 30, 2009

Related U.S. Application Data
(60) Provisional application No. 61/076,717, filed on Jun. 30, 2008.

(51) Int. Cl. C12Q 1/70; C12Q 1/02

(52) U.S. Cl. 435/5; 435/29

(57) ABSTRACT

Hemagglutination assays and hemagglutination inhibition assays were introduced in medical and virology practice more than 60 years ago. Since then, these assays have become important tools for measuring concentrations and strengths of viral cultures, the efficacy of the anti-viral immunization, and for studying the neutralizing capacity of virus-specific antibodies. The present invention comprises an improved hemagglutination inhibition assay (HAI), with at least about a 10-fold increase in sensitivity versus the traditional the HAI, to provide more accurate measurements of components in, for example, fluids from the in vitro MIMIC® system when assessing the effects of anti-viral vaccines (e.g., for seasonal influenza).

### CROSS-REFERENCES TO RELATED APPLICATIONS

This application claims benefit of U.S. provisional patent application No. 61/076,717, filed Jun. 30, 2008, the entire contents of which are incorporated herein by reference.

### BACKGROUND OF THE INVENTION

Viral hemagglutinin proteins agglutinate red blood cells (erythrocytes). This effect provides the basis for virus titration in hemagglutination assays (HA). Specific attachment of antibody to antigenic sites on hemagglutinin molecules interferes with binding between the virus particles and erythrocytes. This effect inhibits hemagglutination and provides the basis for hemagglutination inhibition (HAI or HI) assays.

Hemagglutination assays and hemagglutination inhibition assays were introduced into medical and virology practice more than 60 years ago (Salk (1944) J. Immunol. 49, 87-98). Since that time, they have become important tools for measuring concentrations and strengths of viral cultures, the efficacy of the anti-viral immunization, and for studying the neutralizing capacity of virus-specific antibodies.

Two decades later, attempts were made to develop the method to the degree of a universal standard (Hierholzer et al. (1969) Applied Microbiol. 18, 824-833). However, the protocol for HAI assays kept undergoing minor modifications (e.g., Cross (2002) Seminars in Avian and Exotic Pet Medicine 11, 15-18; Hubby et al. (2007) Vaccine 25, 8180-8189; Wang et al. (2008) Vaccine 26 3626-3633), while preserving the core element intact: visual detection of hemagglutination or hemagglutination inhibition.

In classical HA/HAI assays, the antigen (e.g., live or inactivated virus), either as is, or pre-incubated with an anti-serum or antibody of interest, is mixed with a suspension of purified erythrocytes, such as human group O erythrocytes, or avian, equine, or murine erythrocytes, depending on the type of the viral antigen. After incubation of the mixture in V- or U-bottomed microwells, the visual effect can be two-fold:
If the antiserum is absent or unable to effectively block the virus, the latter links the erythrocytes into a dispersed, three-dimensional agglutinant.
If the antigen is effectively blocked or absent, then the erythrocytes (ERCs) sediment to the bottom of the vial, forming the characteristic bright pellet, or “button.”

To determine the concentration or strength of a viral culture in hemagglutination assays, the sample is subjected to two-fold serial dilutions, until the agglutination vanishes. To determine the efficacy of the antiserum or tested antibody in the HAI assay, the sample is similarly subjected to serial dilution, until agglutination appears. The last dilution before the “borderline” between agglutination/non-agglutination is called the HA or HAI titer.

HA and HAI assays are used for the study of immune responses to a multitude of different pathogenic viruses, including adenoviruses, enteroviruses, reoviruses, myxoviruses, poxviruses, and flaviviruses, that cause a wide spectrum of human and animal illnesses, from influenza and rubella to smallpox and Dengue hemorrhagic fever (e.g., Hatgi et al. (1966) Am. J. Trop. Med. Hyg. 15, 601-610; Hierholzer et al. (1969) Applied Microbiol. 18, 824-833; Cross (2002) Seminars in Avian and Exotic Pet Medicine 11, 15-18; Hubby et al. (2007) Vaccine 25, 8180-8189; Wang et al. (2008) Vaccine 26, 3626-3633). Thus, HA and HAI tests remain major tools in modern virology. Significant improvements to the assays could be of widespread benefit.

While robust, uncomplicated, and reliable, HA and HAI assays lack adequate sensitivity in the cases of some conditions, such as measles, yellow fever, and polyoma (Chapagain et al. (2006) Virology J, 3, 3-5; Fujino et al. (2007) J. Virological Methods 142, 15-20; Niedrig et al. (1999) Trop. Med. Int. Health 4, 67-71). Further, assessments of agglutination are performed by the human eye. Microwells in which ERCs have not been agglutinated by the virus will settle into a red and compact pellet, or “button” at the bottom of the well. Wells in which large-scale agglutination has taken place show a three-dimensional diffuse gel of agglutinated erythrocytes, rather than a compact red pellet. Partially hemagglutinated wells often have the appearance of a halo around the pellet.

In addition to the limited sensitivity of the standard HA/HAI assays with many viruses, as mentioned above, the development of modern in vitro systems for high-throughput analysis of immune responses, such as the MIMIC® system, described in US 2005/0282148, requires improved sensitivity in detection methods. The MIMIC® system is based on cultures of human immune-competent cells developed in a 96-well format, which limits the achievable concentrations and total quantities of the antigen-specific immunoglobulins generated. Thus, there is a continuing need for assays with improved sensitivity, including those based on HA and HAI assays. Such assays could be of great value as well.

### BRIEF SUMMARY OF THE INVENTION

In a first embodiment, the present invention is directed to a hemagglutination assay. The assay comprises: (a) mixing erythrocytes and a sample of an agglutinating virus under conditions permitting agglutination of the erythrocytes, and (b) detecting hemagglutination in the mixture of (a) by light scattering.

In particular aspects, the erythrocytes are human erythrocytes, preferably human group O erythrocytes. In other aspects, the virus is an influenza virus, preferably influenza virus A or an influenza virus B, although any virus that can agglutinate erythrocytes may be used.

The light scattering is detected in this assay using any suitable means for detecting light scattering in a sample. In one aspect, a bead array reader may be used to detect light scattering in the assay.

The assay has a sensitivity for detecting agglutination that is at least about 10-fold greater than the sensitivity of a conventional hemagglutination assay wherein the detection of hemagglutination is by the human eye.

In a further aspect of this embodiment, the assay may be performed using more than one mixture, such as where the assay is repeated with at least one two-fold dilution of the sample of virus, or at least one two-fold dilution of the erythrocytes, or both at least one two-fold dilution of the sample of virus and at least one two-fold dilution of the erythrocytes.

In a preferred aspect of this embodiment, the sensitivity of said assay is increased at least about 10-fold in comparison to a hemagglutination assay wherein the detection of hemagglutination is by the human eye and the detection of the light scattering is performed using a bead array reader.

While the assay of this embodiment can be conducted using a wide range of concentrations and dilutions of the components used in the assay, in one aspect, the concentration of the virus in the final mixture is no greater than about 0.5 hemagglutination units (HAU), such as when influenza H1N1 type virus is used in the assay, and no greater than about 1.0 HAU, such as when influenza H3N2 virus is used. In another aspect, the concentration of the erythrocytes in the final mixture is no greater than a hematocrit of about 0.03%. The assay is quite sensitive and it can be used to detect small amounts of hemagglutination in a sample. In one aspect, the detected amount of hemagglutination in the sample is less than about 10 erythrocytes.

In a second embodiment, the present invention is directed to a hemagglutination inhibition assay. The assay comprises: (a) mixing a sample of an agglutinating virus and an antiserum under conditions permitting binding of an antibody in the antiserum to a hemagglutinin protein on the virus, (b) mixing erythrocytes with the mixture of (a) under conditions permitting agglutination of the erythrocytes, and (c) detecting hemagglutination in the mixture of (b) by light scattering.

In particular aspects, the virus is an influenza virus, preferably influenza virus A or influenza virus B, although any virus that can agglutinate erythrocytes may be used. In other aspects, the antiserum is an antiserum that was raised against the virus, although an antiserum not specifically raised against the virus, but suspected of having the ability to bind viral epitopes, may be used.

In further aspects, the erythrocytes are human erythrocytes, preferably human group O erythrocytes.

The light scattering is detected in this assay using any suitable means for detecting light scattering in a sample. In one aspect, a bead array reader may be used to detect light scattering in the assay.

The assay has a sensitivity for detecting agglutination that is at least about 10-fold greater than the sensitivity of a hemagglutination inhibition assay wherein the detection of hemagglutination is by the human eye.

In a further aspect of this embodiment, the assay may be performed using more than one mixture, such as where the assay is repeated with at least one two-fold dilution of the sample of virus, at least one two-fold dilution of the sample of antiserum or at least one two-fold dilution of the erythrocytes, or both at least one two-fold dilution of the sample of virus and at least one two-fold dilution of the sample of antiserum.

In a preferred aspect of this embodiment, the sensitivity of said assay is increased at least about 10-fold in comparison to a hemagglutination inhibition assay wherein the detection of hemagglutination is by the human eye and the detection of the light scattering is performed using a bead array reader.

While the assay of this embodiment can be conducted using a wide range of concentrations and dilutions of the components used in the assay, in one aspect, the concentration of the virus in the final mixture is no greater than about 0.5 HAU, such as when influenza H1N1 type virus is used, and about 1.0 HAU, such as when influenza H3N2 virus is used. In another aspect, the concentration of the erythrocytes in the final mixture is no greater than a hematocrit of about 0.03%.

The assay is quite sensitive and it can be used to detect small amounts of hemagglutination in a sample. In one aspect, the detected amount of hemagglutination in the sample is less than about 10 erythrocytes.

In a third embodiment, the present invention is directed to a method of detecting hemagglutination in a sample, comprising detecting hemagglutination in a sample by light scattering.

The light scattering is detected in this assay using any suitable means for detecting light scattering in a sample. In one aspect, a bead array reader may be used to detect light scattering in the assay.

The assay has a sensitivity for detecting agglutination that is at least about 10-fold greater than the sensitivity of a hemagglutination assay or a hemagglutination inhibition assay wherein the detection of hemagglutination is by the human eye.

The assay is quite sensitive and it can be used to detect small amounts of hemagglutination in a sample. In one aspect, the detected amount of hemagglutination in the sample is less than about 10 erythrocytes.

### BRIEF DESCRIPTION OF THE FIGURES

FIG. 1. Light-scattering effects observed reading human erythrocytes in the BioPlex® bead array reader. BioPlex®-assisted registration of the light-scattering objects in samples of human group O erythrocytes (6.25×10⁶ cells/mL, ˜0.1% HCT), with (panel A) and without (panel B) influenza virus (H1N1 Solomon Islands BPL-inactivated influenza virus, CDC standard, CDC HA titer: 160). Human erythrocytes 60 μL; virus diluted 1:320, 30 μL; media, 30 μL; sampling, 80 μL; 2016 “events” registered. Left side of panels: Discrimination panels showing histograms of light-scattering objects. Right side of panels: Classification panels showing scattering objects (dots) towards the BioPlex® registration regions (pale ovals).

FIG. 2. Effect of inactivated influenza virus and anti-influenza human serum on the number of light-scattering objects registered in the BioPlex® bead array reader. Concentrations of the erythrocytes are the same as in FIG. 1. FIG. 2A: gated light scattering events in BioPlex® in the presence of human erythrocytes and influenza virus: 30 μL of four dilutions of H1N1 Solomon Islands influenza virus (CDC standard, CDC HA titer: 160), 30 μL of media, 60 μL of either media (front two rows), or human erythrocytes diluted to 0.1% HCT (back two rows). FIG. 2B: BioPlex® HAI test; 30 μL of the media or anti-influenza serum (donor #419, post-immunization serum); 30 μL of either media or virus diluted 1:320, 60 μL of erythrocytes diluted to 0.1% HCT.

FIG. 3. Human group O erythrocytes used in the BioPlex® bead array experiments, as seen on a hemacytometer. FIG. 3A, without virus. FIG. 3B, with H1N1 Solomon Islands virus, diluted 1:20.

FIG. 4. Conditions, protocol, and typical raw results of an example BP-HAI experiment. The virus used was H1N1 Solomon Islands BPL-inactivated influenza virus (CDC standard, CDC HA titer: 160).

FIG. 5. Conditions, protocol, and typical raw results of an example BP-HA experiment. Erythrocyte (ERC) concentrations and volumes are the same as in FIG. 4. OVA, grade V, was used in the PBS buffer, instead of BSA.

FIG. 6. Mathematical modeling of BP-HAI and determining BP-HAI titers using the curves “Serum Dilution-BP Events.” FIG. 6A shows use of the low affinity curve. FIG. 6B shows use of the high affinity curve.

FIG. 7. A typical layout for an example BP-HAI experiment.

FIG. 8. BP-HAI titers for a sub-pool of 15 human sera with H1N1 Solomon Islands virus, plotted versus regular HAI titers, with high affinity calculations shown in FIG. 8A, and low affinity calculations in FIG. 8B. The table in FIG. 8B contains pool-averaged BP-HAI sub-titers (i.e., D_{1/2} values) and standard errors.

FIG. 9. BP-HAI titers versus regular HAI titers for an extended pool of 33 human sera with the H1N1 Solomon Islands influenza virus. The insert from the right side demonstrates reproducibility of the BP-HAI titration for two randomly selected sera. The table shows virus dilutions used in the experiments, showing sensitivity improvement in the BP-HAI versus regular HAI.

FIG. 10. BP-HAI titers versus regular HAI titers for an extended pool of 34 human sera with H3N2 Wisconsin influenza virus. The insert from the bottom shows the reproducibility of the BP-HAI titration for six randomly selected sera. The table shows virus dilutions used in the experiments, showing sensitivity improvement in the BP-HAI versus regular HAI.

FIG. 11. BP-HAI titers versus regular HAI titers for the pool of 30 MIMIC® samples with the H1N1 Solomon Islands influenza virus. The table shows virus dilutions used in the experiments, showing sensitivity improvement in the BP-HAI versus the regular HAI. The virus dilution used in the regular HAI assays for the MIMIC® samples was four times higher than for the donor sera (160 vs. 40).

FIG. 12. Agglutinating capacity of the avian influenza virus (H5N1) in comparison with other influenza viruses (FIG. 12A) and blocking of the avian influenza virus with anti-influenza A polyclonal antibodies demonstrated by the BP-HAI (FIG. 12B). Polyclonal goat anti-influenza A antibodies used in the experiment were raised against the H1N1 USSR-1999 influenza strain.

FIG. 13. Blocking of the avian influenza virus with sera from donors immunized against seasonal influenza.

### DETAILED DESCRIPTION OF THE INVENTION

The following abbreviations are used in the text: BP, BioPlex® bead array reader; BSA, bovine serum albumin; ERC, erythrocytes (red blood cells); HA, hemagglutination assay; BP-HA, BioPlex®-assisted hemagglutination assay; HAI, or HI, hemagglutination inhibition assay; BP-HAI, BioPlex®-assisted hemagglutination inhibition assay; HCT, hematocrit, the proportion of blood volume that is occupied by red blood cells; MIMIC®, modular immune in vitro construct, an automated, high-throughput system for testing, for example, the immunogenicity of vaccines and drug compounds, developed by VaxDesign Corp.; OVA, ovalbumin, from chicken eggs; PSG, penicillin-streptomycin-glucose standard mix.

The present invention is based on the discovery that devices that can determine light scattering in a sample, such as a biological sample, can be used to detect small quantities of hemagglutination, such as agglutinations of erythrocytes by some viruses, such as influenza viruses. This discovery has led to the development of the improved hemagglutination assays and hemagglutination inhibition assays of the present invention.

The improved hemagglutination assays and hemagglutination inhibition assays show a great increase in sensitivity in comparison to conventional approaches, especially assays where the human eye is used to determine the result. Additionally, the improved assays of the present invention can be conducted in a high-throughput manner (the BioPlex® reader can handle standard 96-well plates), they uses smaller sample volumes (blood, reagents, and anti-serum), and they do not depend on a qualitative “reading” of the results by the human eye. Further, the improved assays can be readily automated.

In one embodiment, the present invention is directed to a hemagglutination assay (HA). The hemagglutination assay comprises: (a) mixing erythrocytes and a sample of an agglutinating virus under conditions permitting agglutination of the erythrocytes, and (b) detecting hemagglutination in the mixture of (a) by light scattering.

In another embodiment, the present invention is directed to a hemagglutination inhibition (HAI) assay. The HAI assay comprises: (a) mixing a sample of an agglutinating virus and an antiserum under conditions permitting binding of an antibody in the antiserum to a hemagglutinin protein on the virus, (b) mixing erythrocytes with the mixture of (a) under conditions permitting agglutination of the erythrocytes, and (c) detecting hemagglutination in the mixture of (b) by light scattering.

In a third embodiment, the present invention is directed to a method of detecting hemagglutination in a sample, comprising detecting hemagglutination in a sample by light scattering.

In each of the embodiments of the present invention, the techniques, reagents and materials used in performing the agglutinations may be the techniques, reagents and materials used in conventional hemagglutination assays and hemagglutination inhibition assays, such as those taught in WHO Manual on Animal Influenza Diagnosis and Surveillance, WHO/CDS/CSR/NCS2002.5 Rev. 1. Exemplary reagents and materials are provided as follows.

The erythrocytes that may be used in the HA and HAI assays of the present invention are preferably human erythrocytes from any blood groups (A, B, AB or O). In a more preferred aspect, human group O erythrocytes are used in the improved assays of the present invention. The skilled artisan will understand that the concentration of erythrocytes used in a particular assay will depend on a number of different factors, such as the source of the erythrocytes, sample volume and the source and concentration of the other components being used in an assay. In one aspect, the hematocrit of the final mixture of erythrocytes, virus and/or antiserum is a hematocrit of no greater than about 0.01%, 0.02%, or 0.03%.

The virus that may be used in the assays of the present invention may be any virus that bears hemagglutinin protein that can agglutinate erythrocytes. The viruses may naturally encode the hemagglutinin protein, or may be a virus engineered to express such a protein. In one aspect, the virus is an adenovirus, enterovirus, reovirus, myxovirus, poxvirus, or flavivirus. In another aspect, the virus is an influenza virus, including influenza virus A and influenza virus B. Specific examples include H1N1 Solomon Islands influenza virus, H3N2 Wisconsin influenza virus, and avian H5N1 Vietnam influenza virus. In one aspect, the concentration of the virus in the final mixture is no greater than about 0.5 hemagglutination units (HAU), such as when influenza H1N1 type virus is used, or about 1.0 HAU, such as when influenza H3N2 virus is used.

The skilled artisan will understand that the concentration of virus used in a particular assay will depend on a number of different factors, such as the identity of the virus, sample volume, and the source and concentration of the other components being used in an assay. Further, the concentration of the virus to be used in an assay will generally be based on the known value of the lowest dilution of the virus at which hemagglutination occurs in a conventional HA assay. This dilution is considered to be 1 hemagglutination unit (HAU). Higher and lower concentrations of virus, based on 1 HAU, may be used as starting points in an assay of the present invention. For example, if a 1:128 dilution is the lowest dilution of the virus at which hemagglutination occurs in a conventional HA assay, dilutions of 1:128, 1:256, 1:512, 1:1024, 1:2048, 1:4096 and 1:8192 may be used. The skilled artisan will understand that any dilution of virus may be used, or any series of dilutions, whether based on a factor of 2, or some other number.

The hemagglutinin protein on the virus envelope may be a naturally occurring protein, or it may be genetically engineered. Examples of influenza hemagglutinin proteins include subtypes H1 through H16 produced by influenza viruses. The genetically engineered hemagglutinin proteins may include mutations that provide enhanced or inhibited activities to the protein, in comparison to a wild-type version of the protein. The skilled artisan will recognize that the assays of the present invention may be performed using solely hemagglutinin proteins and that the entire virus particle is not required.

The antiserum that may be used in the assays of the present invention may be any antiserum that contains antibodies that bind to one or more epitopes on a virus having the ability to agglutinate erythrocytes, or suspected of having the ability to agglutinate erythrocytes (in the case of an antiserum being tested in the HAI). Alternatively, or in addition, the antiserum may be any antiserum that contains antibodies that bind to one or more epitopes of a hemagglutinin protein, or suspected of containing antibodies having the ability to bind to one or more epitopes of a hemagglutinin protein (in the case of an antiserum being tested in the HAI). The antiserum may be serum obtained from a living source, such as a human, bird, horse, rabbit, mouse, goat, pig, guinea pig, or rat. The living source of the antiserum may have been immunized with a particular antigen, such as a hemagglutinin protein, although the living source need not have been specifically exposed to the antigen. The antiserum may also be a serum produced in vitro to comprise antibodies that bind to a virus or a hemagglutinin protein. The antibodies may be monoclonal or polyclonal. Further, the antibodies may be full length, or an antigen binding fragment, such as Fab and F(ab)₂ fragments and single chain antibodies. The antibodies may also be naturally occurring antibodies, humanized antibodies or chimeric antibodies. Any antibody that binds to an agglutinating virus or a hemagglutinin protein, whatever the source, may be used in the assays.

The skilled artisan will understand that the concentration of the antisera used in a particular assay will depend on a number of different factors, such as the source of the antisera, the type of antibody in the antisera, the affinity of the antibodies in the antiserum, the concentration of non-antibody components of the antisera, the sample volume, and the source and concentration of the other components being used in an assay. Further, the concentration of the antisera to be used in an assay can be in some cases based on the known value of the lowest dilution of the antisera at which the antibodies in the antisera can block hemagglutination from occurring in a conventional HAI assay. Higher and lower concentrations of antisera may be used as starting points in an assay of the present invention based on this dilution. For example, if a 1:128 dilution is the lowest dilution of the antisera at which hemagglutination can be blocked in a conventional HAI assay, dilutions of 1:512, 1:1024, 1:2048, 1:4096 and 1:8192 may be used. The skilled artisan will understand that any dilution of antisera may be used, or any series of dilutions, whether based on a factor of 2, or some other number. In one aspect, the concentration of the antibody in the final mixture of an assay may be about 0.1, 0.5, 1, 2, 3, 4, 5, 6, 7, 8, 9 or 10, 20, 30, 40, or 50 nM. In such aspects, the affinity of the antibodies in the antisera for a viral epitope (K_{diss}) is about 0.01, 0.05, 0.1, 0.5, 1, 5, 10, 20, 30, 40 or 50 nM.

The light scattering is detected in this assay using any suitable means for detecting light scattering in a sample. In one aspect, a bead array reader may be used to detect light scattering in the assay. The skilled artisan will understand that light scattering is detected as the sample in the device actively flows by a detector. For example a BioPlex® bead array reader or a Luminex® machine, or any other device that operates on a similar principle, may be used. Light scattering spectrometers may also be used. Bead array readers are preferable where high throughput is desired. While devices such as the BioPlex® bead array reader are generally used to measure the fluorescence of an object in a sample, the fluorescence-measuring abilities are not utilized in the assays of the present invention.

When using devices such as the BioPlex® bead array reader for the HA/HAI assay, normally no change to the standard settings is required. In a preferred embodiment, the discrimination gates settings were: low border, 4335; high border, 10000. The sample timeout was set at 30 s; the sample size is preferably 60 μL. In some experiments, gate settings can be changed to cut off undesired light scattering objects, such as debris or large clumps of erythrocytes.

The assays of the present invention have greater sensitivity in detecting agglutination than conventional HA and HAI assays. In one aspect, the assays of the present invention have at least a 5-fold greater sensitivity in detecting agglutination than conventional HA and HAI assays. That is, the assays of the present invention are at least 5-fold more sensitive than conventional assays, such as those using the human eye. In some aspects, the assays of the present invention have at least a 10, 25, 50, or 100-fold greater sensitivity in detecting agglutination than conventional HA and HAI assays. While the size of the agglutination that may be detected using the assays of the present invention will vary based on the components used in the assay, including the source of the erythrocytes, and the identity of the antisera and the virus, as discussed below an agglutination of as few as two erythrocytes may be detected using the assays. In particular aspects, agglutinations of less than about 3, 4, 5, 6, 7, 8, 9, 10, 20, 25, 30, 50, 75, or 100 erythrocytes may be detected using the assays of the invention.

In further aspects of each of the embodiments of the present invention, the assays may be performed using one mixture or a series of more than one mixture. In the case of the former, for example, a simple HA assay may be performed using one test virus to assay its ability to agglutinate erythrocytes at a selected concentration. In a variation, a HA assay may be performed using a number of different test viruses, each at the same concentration, to determine their ability to agglutinate erythrocytes at the selected concentration. In the case of the latter, the ability of a test antiserum to block agglutination may be assayed by using a number of different dilutions of the antiserum. In further examples, the assays may be repeated with serial dilutions of the sample of virus, or serial dilutions of the erythrocytes, or serial dilutions of the antisera, or any combination thereof.

As will be evident from the detailed description above, and the examples to follow below, the present invention comprises improved hemagglutination assays and hemagglutination inhibition assays, using a bead array reader to detect erythrocyte agglutination, with at least about a 10-fold increase in sensitivity versus conventional hemagglutination assays and hemagglutination inhibition assays. The improved assays provide more accurate measurements of components in, for example, fluids from the MIMIC® system, when assessing the effects of anti-viral vaccines (e.g., those against seasonal and avian influenza).

It has been have found that ordinarily the vast majority of erythrocytes are not registered as legitimate light-scattering objects, or “events,” in a bead array reader (e.g., BioPlex® or Luminex® machines). However, the number of registered objects significantly increases in the presence of, for example, influenza virus (inactivated or otherwise), admixed with the erythrocytes prior to reading. Thus, micro-clusters (e.g., doublets, triplets, quadruplets) of agglutinated erythrocytes can be readily detected by their light-scattering properties in, for example, a BioPlex® bead array reader.

Pre-incubation of, for example, influenza virus with anti-influenza serum significantly reduces the number of “events” detected. Anti-influenza sera block the formation of the micro-clusters of erythrocytes (if antibodies in the serum bind to HA) and, thus, reduce the signal detected by the bead array reader (unclustered erythrocytes are not read, except at a very low background noise level). The dilution at which the antiserum stops affecting the reduction is related to the amount of antibody present and the affinity of the antibody. As a control, an aliquot of the same virus without erythrocytes produces no detectable objects in the bead array reader.

The BioPlex®-based HAI and HA (BP-HA and BP-HAI) methods of the present invention demonstrated consistent results on inactivated H1N1 Solomon Islands, H3N2 Wisconsin virus, avian H5N1 Vietnam virus, human anti-influenza sera, and MIMIC® supernatant samples. A typical BP-HAI assay takes ˜2 h of total incubation and reading time, and the BP-HA assay takes ˜1 h 20 min, comparable with traditional HAI and HA assays. In particular examples, the BP-HAI and BP-HA assays use human group O erythrocytes at the level of ˜0.1% hematocrit before mixing with virus and sera, and ˜0.033% hematocrit after mixing, which is about 15 times lower than in the traditional HAI assay with human erythrocytes (˜1.5% HCT before mixing with virus and sera). BP-HAI titers for a pool of 33 human anti-influenza sera demonstrated an excellent correlation with the titers obtained with a traditional HAI: the correlation coefficients, K_{corr}>0.99. The BP-HAI with H1N1 Solomon Islands influenza virus demonstrated an average sensitivity ˜20 times better than the regular HAI. The BP-HA titration showed approximately 15 times better sensitivity than the regular HA assay. The BP-HAI with H3N2 Wisconsin virus has demonstrated average sensitivity ˜10 times better than the regular HAI. BP-HAI can provide direct measurements of components in MIMIC®fluids without any need to concentrate the samples.

### EXAMPLES

### Example 1

### Using a Bead Array Reader for the Detection of Hemagglutination

Human group O erythrocytes were separated from fresh blood. Erythrocytes were washed three times in Dulbecco PBS buffer from lymphocytes and plasma using centrifugation, then washed twice with complete RPMI media containing 1% PSG additive (centrifugation at 350 g, room temperature), and resuspended in the same media at 10% hematocrit (HCT). The erythrocyte suspension was stored in 0.5-mL aliquots at 4° C. Human anti-influenza sera were obtained from donors participating in the 2007-2008 influenza screening program and collected at the Florida Blood Center. Sera samples were sealed and kept at 4° C. for short-term storage or at −80° C. for long-term storage. H1N1 Solomon Islands BPL-inactivated influenza virus was obtained from the US Centers for Disease Control and Prevention (CDC), Atlanta, Ga. Bovine serum albumin, heat shock-separated, low endotoxin, was obtained from Sigma-Aldrich (Cat. #A9430). Chicken ovalbumin, grade V, was from Sigma-Aldrich (Cat. #A5503). A BioPlex® reader (BioPlex®-100, Bio-Rad) was used as a light scattering discrimination device, without using its fluorescence measurement function.

The BioPlex® reader displays the following information panels:

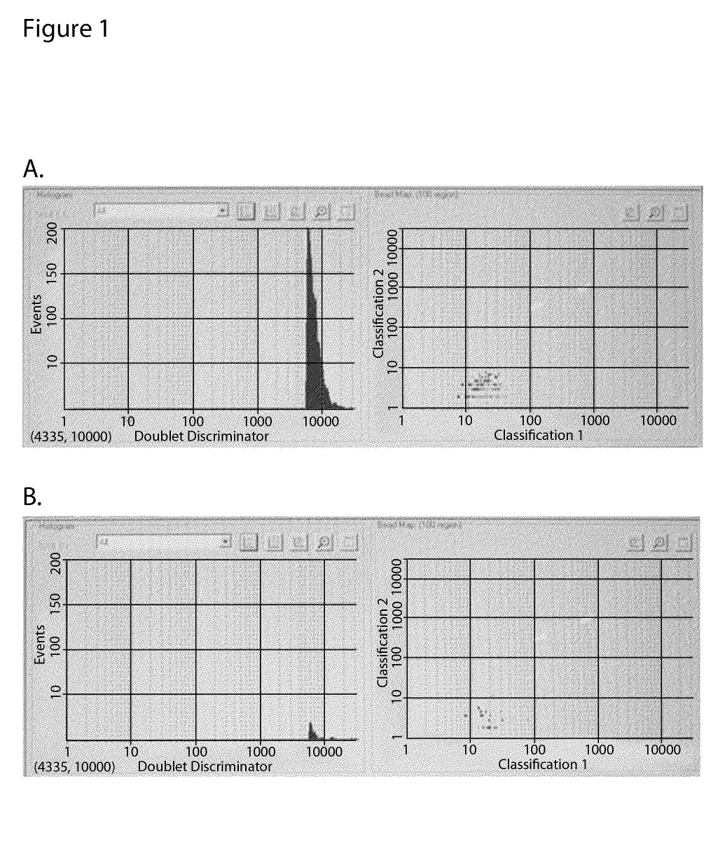

1. The discrimination panel, showing the number of particulate objects in a sample, registered by the light scattering detector, with results shown graphically as a histogram (registration and counting of individual beads and agglomerates; black histograms on the left in the panels of FIGS. 1A and 1B), and

2. The classification panel, showing positioning of the registered particulate objects in the two-dimensional fluorescence map (recognition of fluorescence-coded Luminex beads used in regular bead array experiments; dot clusters on the right in the panels of FIGS. 1A, 1B).

It was found that the vast majority of erythrocytes flowing through the detection chamber of the BioPlex® were not registered as legitimate objects, or “events.” The experiment with bare erythrocytes (no virus) presented in the panel of FIG. 1B showed 96 registered “events” per approximately 2.5×10⁵ erythrocytes that passed the detector. On the classification map, the registered objects were observed far away from the map regions designated for the Luminex fluorescent-coded beads (white ovals in the middle of the map represent randomly chosen regions #27 and #45). Apparently, the fluorescence of the registered objects could not be measured by the BioPlex® reader in this experiment, which was not a concern because the BioPlex® reader was actually being used as a light scattering detector only.

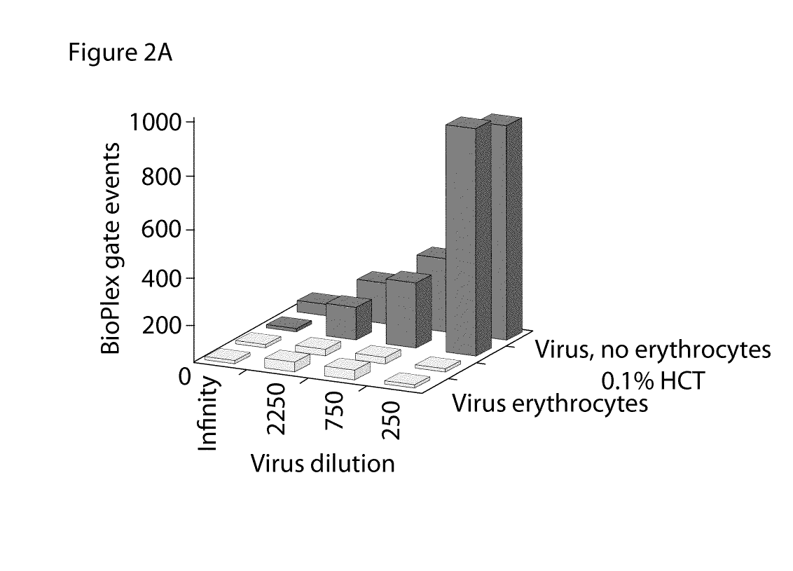

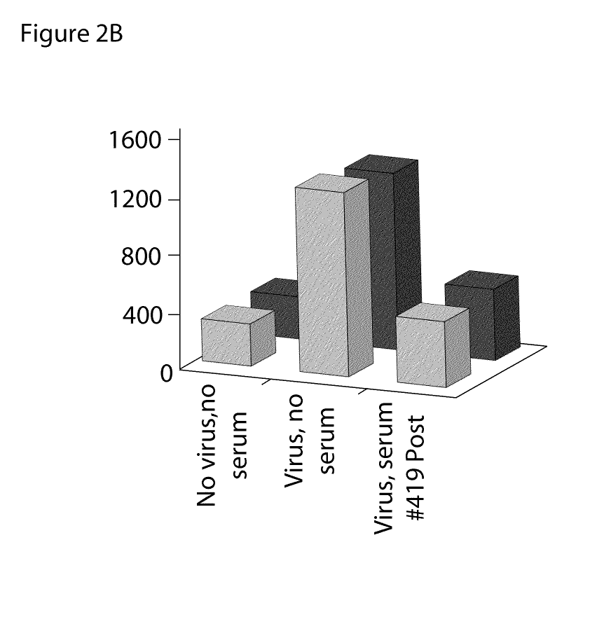

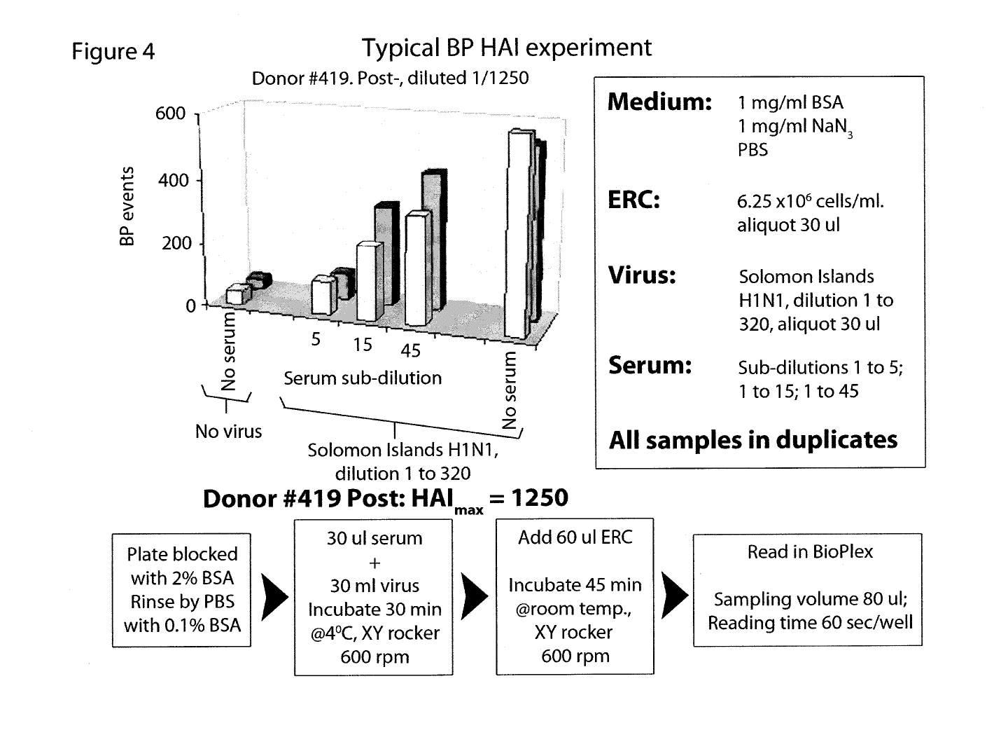

The number of the registered objects increased significantly in the presence of H1N1 Solomon Islands influenza virus admixed with human ERC before reading (compare the left panels in FIGS. 1A and 1B; compare the front two rows with the back two rows in FIG. 2A). However, if the virus was pre-incubated with human anti-influenza serum, the number of the detected light-scattering “events” decreased again (compare “Virus, no serum” and “Virus, serum #419 Post” in FIG. 2B; where “serum #419 Post” is human anti-influenza serum). At the same time, aliquots of the bare virus passed through the BioPlex® without erythrocytes did not produce any detectable objects above the regular and insignificant background level (FIG. 2A, front two rows). The conditions for these experiments are described in more details in FIG. 4.

### Example 2

### Nature of the Light Scattering Objects Detected a Bead Array Reader in Human Erythrocytes Mixed with Virus

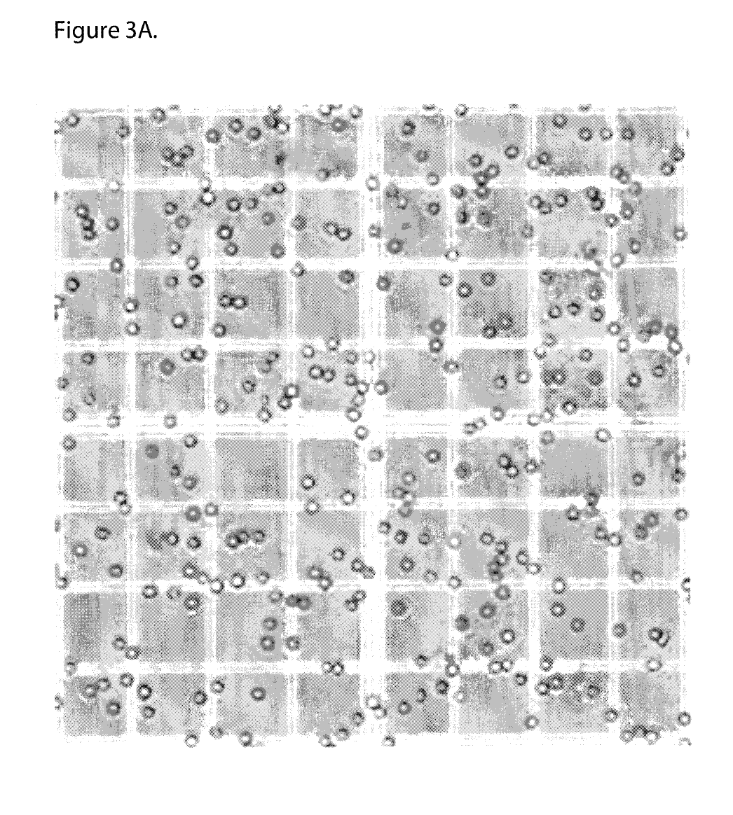

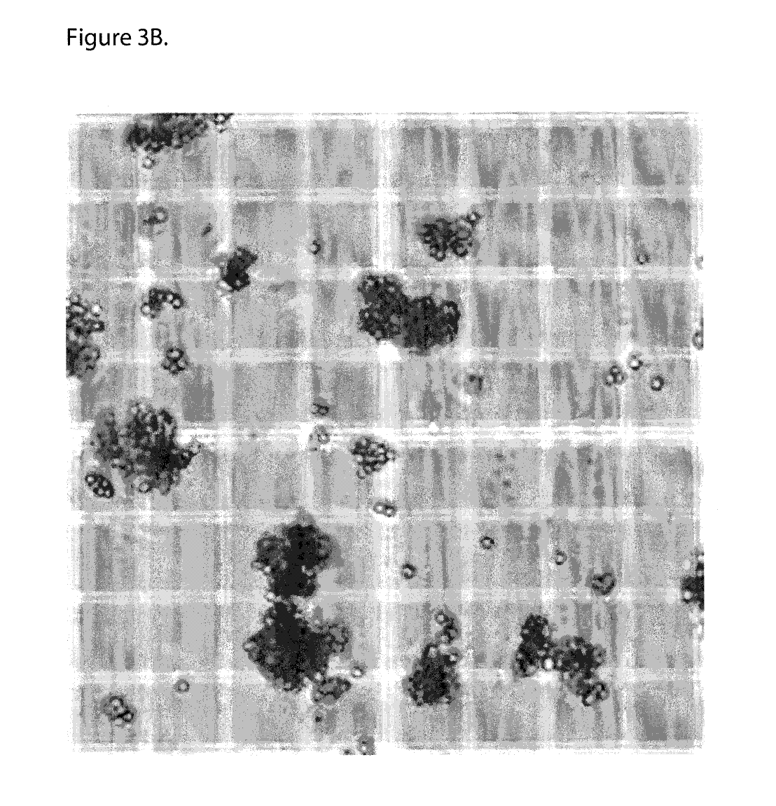

The samples used in the experiment shown in FIG. 1 were examined with a hemacytometer. Erythrocytes cultured without virus were largely dispersed, with a small number of doublets and triplets (FIG. 3A). On the other hand, in the presence of the H1N1 Solomon Islands influenza virus, the erythrocytes quickly formed significant clusters (FIG. 3B), the latter being an expected manifestation of hemagglutination at the reduced concentration of erythrocytes.

However, the appearance of these large clusters failed to explain the results of the experiment in all details. Indeed, the shapes of the light scattering diagrams, with and without the presence of the virus, were similar (FIGS. 1A and 1B, panels on right) while the sample without virus (erythrocytes only) showed no large clusters at all. When the sample containing the erythrocytes and virus was pushed through a thin capillary similar to that used in BioPlex® reader, before placing it on a hemacytometer, large clusters were noticed not immediately, but only after a delay of a few seconds (data not shown). Low concentrations of erythrocytes favor the formation of smaller clusters, simply for kinetic reasons. Erythrocytes with no virus still give a low level of “events” on the BioPlex® reader (a “background” level), although erythrocytes without viruses contain no clusters that can be found using a hemacytometer (data not shown). The histogram of the “events” for the bare erythrocytes is similar to the histogram obtained for the (erythrocytes+virus) system, indicating that the objects that produce “events” were similar in both cases. The only kinds of objects that could produce “events” in bare erythrocytes are occasional doublets and triplets of erythrocytes; they may link in doublets and triplets due to statistical collisions.

These observations lead to the conclusion that the light-scattering objects registered by the bead array reader in the samples of human erythrocytes were actually micro-clusters, such as doublets, triplets, or quadruplets of agglutinated erythrocytes, rather than large clusters.

### Example 3

### Development of Bead Array Reader-Based HAI and HA Assays

Experimental conditions, the protocol and typical results of a typical BP HAI assay are shown in FIG. 4. The concentration of erythrocytes was ˜6.3×10⁶ cells/mL, corresponding to ˜0.1% hematocrit (HCT) before mixing with virus and sera. The dilution of the virus was adjusted to 1 to 320, to compare with 1 to 40 used for the same virus in the conventional HAI.

Human sera were initially diluted to the titer of the last HAI data obtained in a conventional HAI assay performed earlier (for a suitable protocol, see WHO Manual on Animal Influenza Diagnosis and Surveillance, WHO/CDS/CSR/NCS2002.5 Rev. 1). For the example shown in FIG. 4, the HAI titer obtained with the conventional HAI assay was 1250. Thus, before testing in the BP-HAI, the serum was pre-diluted 1 to 1250. Subsequently, samples of the pre-diluted sera were further sub-diluted 5, 15, and 45 times, to provide a range of dilutions of about one log.

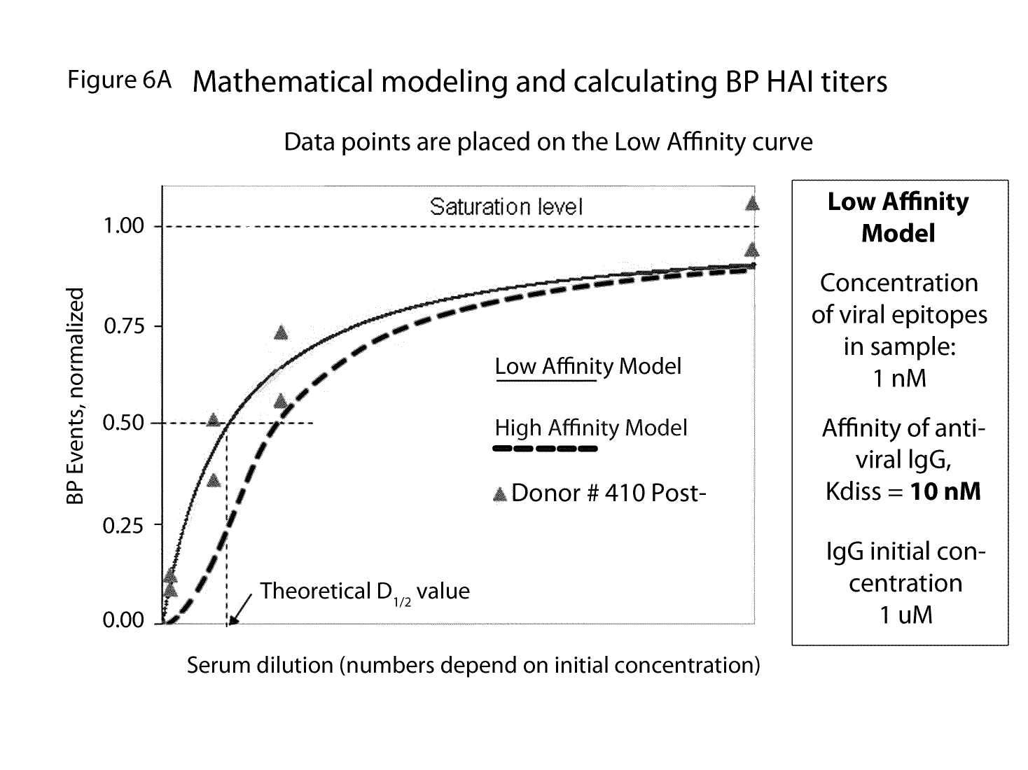

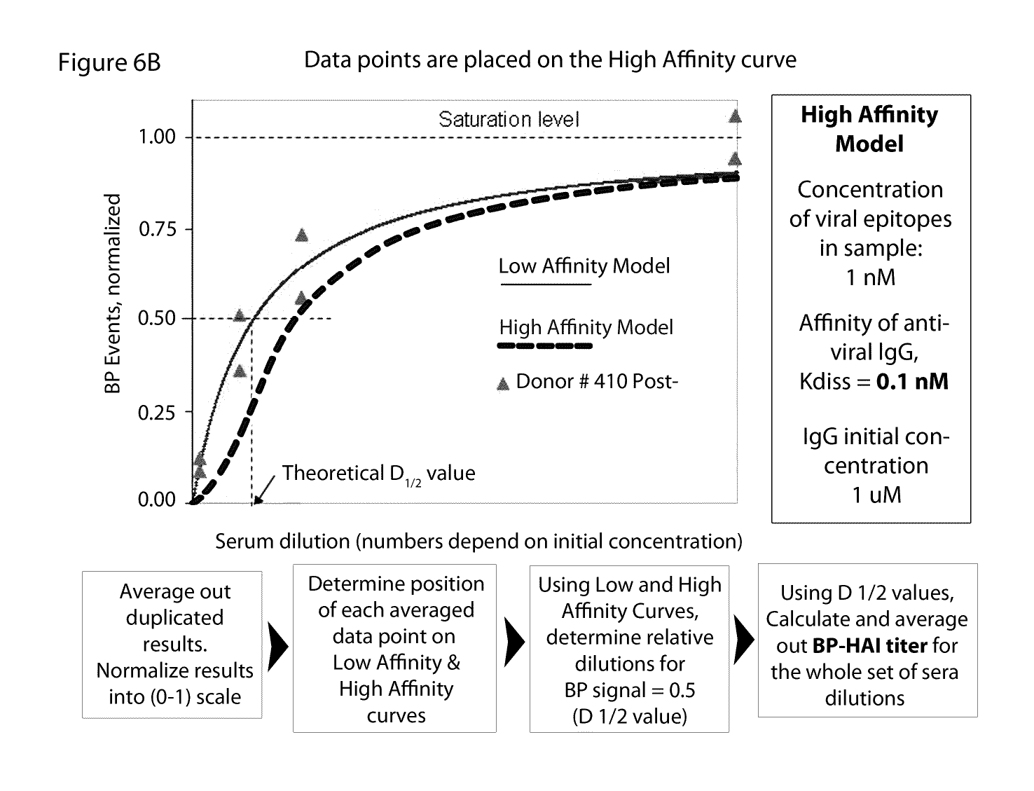

After the steps shown in FIG. 4 were performed as described, the approximate BP-HAI titer could be obtained directly from the registered numbers of “events” detected by the BioPlex® reader, at different sub-dilutions of the serum, by interpolation. For example, for the results displayed in the graph in FIG. 4, the BP HAI sub-titer was apparently somewhat higher than 15, but significantly lower than 45. However, for better precision, mathematical modeling and a data processing algorithm were developed (Example 4; FIG. 6). The final BP-HAI titer determined in the described experiment is a product of the initial dilution (e.g., 1250), and the newly determined sub-titer (a number interpolated between subdilutions 15 and 45).

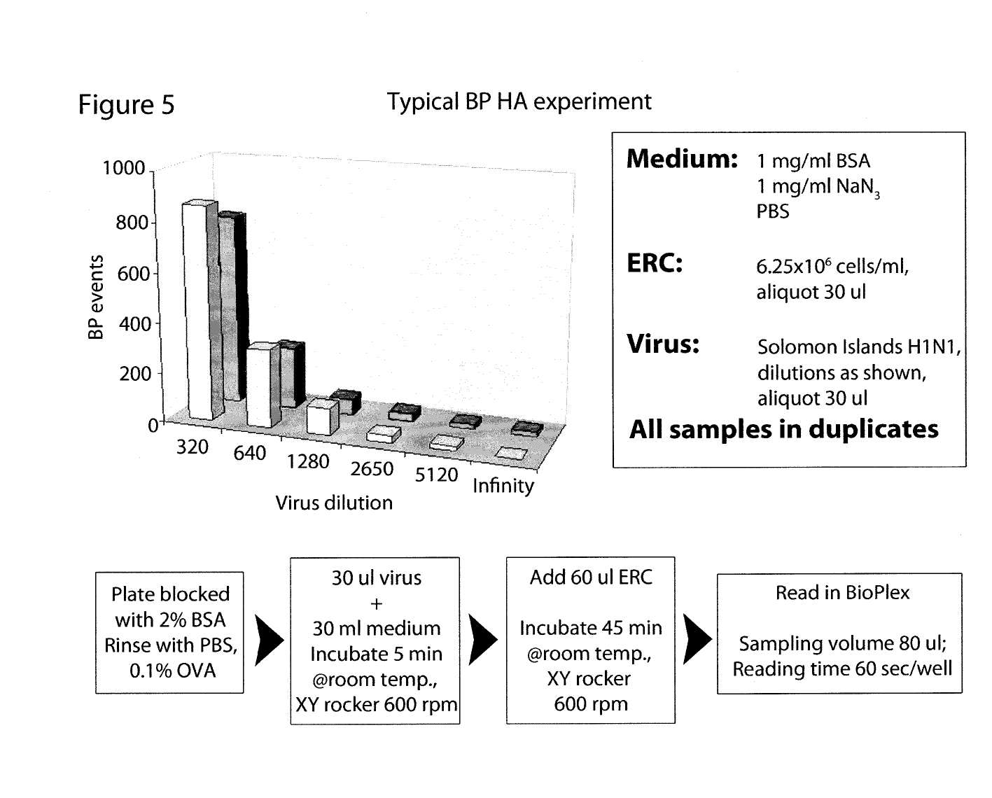

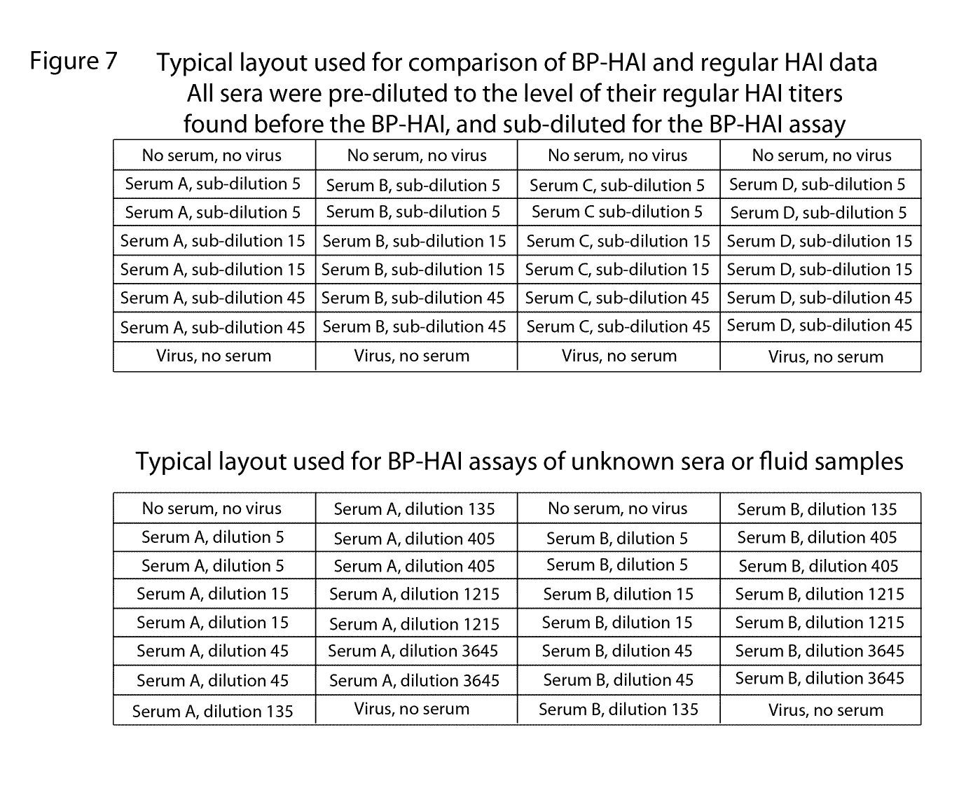

Pre-dilution of the tested sera to their regular HAI titer levels was used for the demonstrational and test experiments only, where the regular HAI and the BP-HAI were compared side-to-side to show the correlation between the two methods and to estimate the improvement in sensitivity provided by the BP-HAI. When blinded samples of sera or experimental fluids were tested, the BP-HAI titers were determined via straightforward serial dilutions, in a way similar to the regular HAI (FIG. 7, lower table). The experimental conditions, the protocol, and typical results of an example HA assay experiment are shown in FIG. 5. The concentration of erythrocytes and most of the details of the protocol remained the same as for the HAI assay, except that high-purity grade V ovalbumin was used in the diluting media, instead of BSA

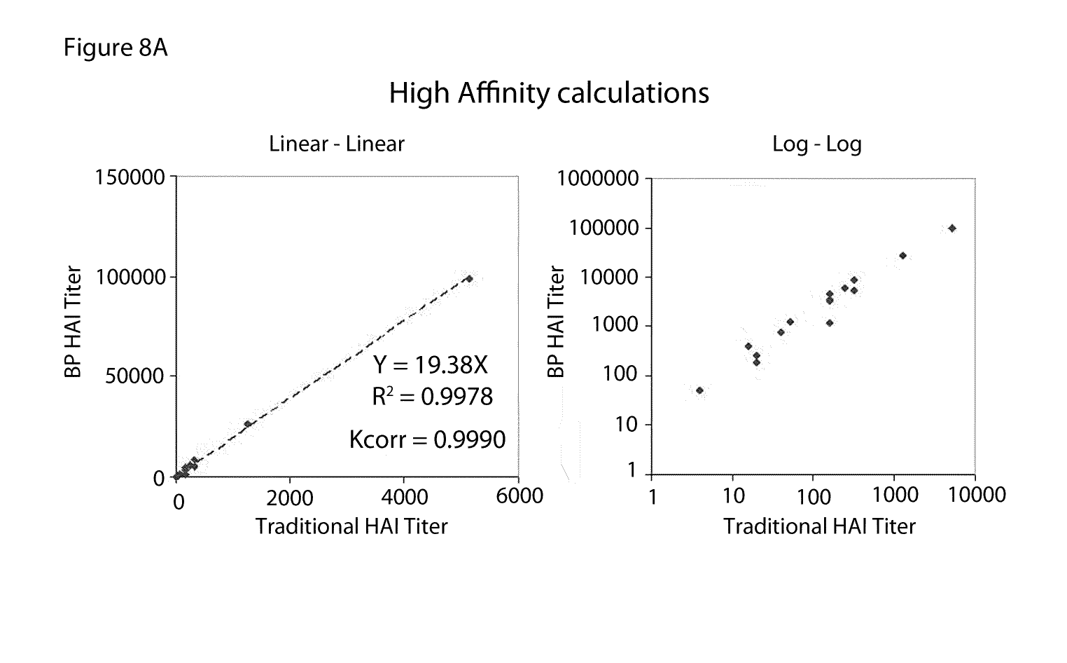

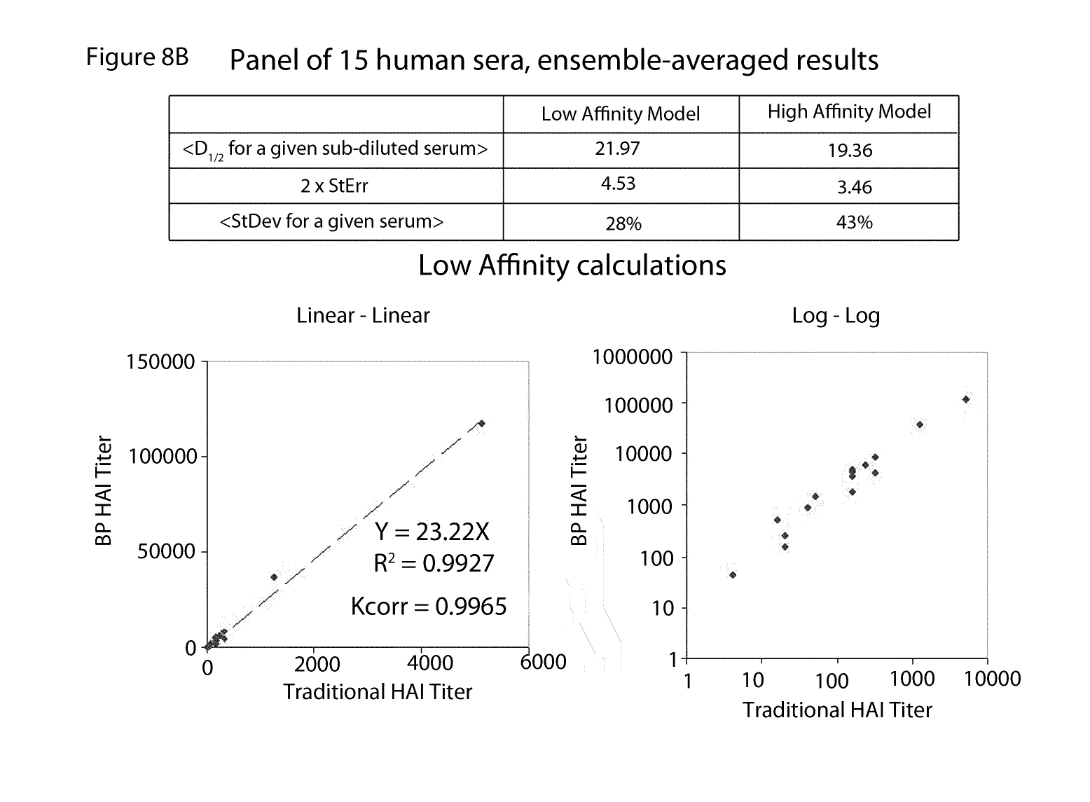

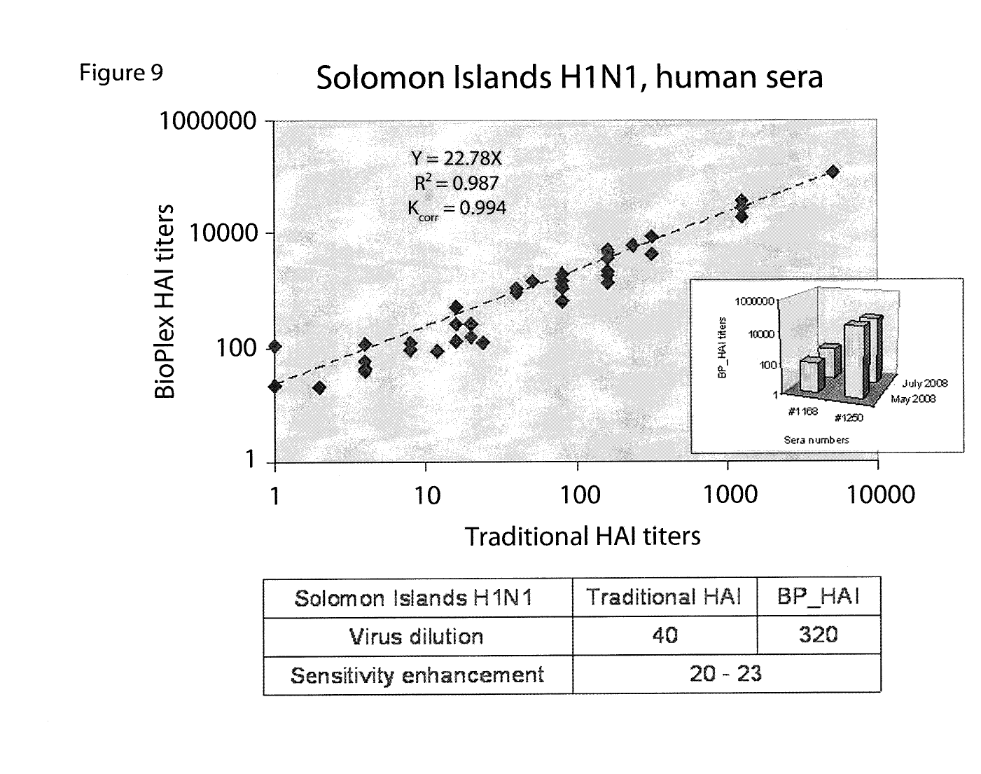

Remembering that the regular HAI gave a titer of 1250 for the serum used in the FIG. 4 experiment, and the classical HA assay gave a HA titer of 60 for the virus used in the experiments shown in both FIGS. 4 and 5, a raw estimate of the improvement in sensitivity for the BP-HAI was ˜15-25-fold, and for BP-HA assays was ˜20-fold. Scaled up BP HAI experiments performed subsequently with a panel of 15 human sera (FIG. 8) and 33 human sera (FIG. 9) and the Solomon Islands H1N1 influenza virus demonstrated improvement of sensitivity for the BP HAI versus traditional HAI of ˜22-23-fold.

### Example 4

### Mathematical Modeling of the BP-HAI

More precise values of BP-HAI titers can be obtained via mathematical modeling of the HAI, and using the model curves “Serum Dilution-BP Events” for calculating sub-titers and final BP-HAI titers of a tested sera (FIGS. 6A, 6B).

The basic assumptions for the mathematical model were the following:

1. The HAI effect is directly proportional to the number of virus surface epitopes blocked with antibodies;

2. The affinity of all the anti-virus antibodies was considered equal to an average affinity of the whole antibody pool, and the antibodies were considered monovalent. Thus, possible two-valent attachments of the antibodies were not taken into consideration, as being less probable.

Two extreme variants of the HAI system were considered:

1. A high-affinity model (K_{diss} for the antibodies lower than the concentration of the viral epitopes);

2. A low-affinity model (K_{diss} for the antibodies higher than the concentration of the viral epitopes).

The normalized (i.e., brought to a 0-1 scale) curves “Serum Dilution-BP Events” for both low- and high-affinity variants are shown in FIGS. 6A and B, together with the lists of numeric parameters used for modeling, along with the scheme of the algorithm used for calculating the BP-HAI titer. High- and low-affinity variants resulted in different shapes of the model curves “Dilution-BP events,” sigmoid and convex respectively. It was found that these characteristic shapes persisted over wide ranges of initial serum dilutions and basic numerical values used for modeling (data not shown).

To calculate the BP-HAI titer of the tested serum, it was sufficient to perform the following:

1. Average out and normalize (i.e., bring to a 0-1 scale) the duplicate BP-HAI results for each serum sub-dilution 5, 15, and 45,

2. Fit the averaged results for the sub-dilutions 5, 15, and 45 using a standard list square procedure.

3. Determine the effective midpoint sub-dilution, or sub-titer, D_{1/2}, that corresponds to 50% of the maximal BP events found in the experiment, and determine the standard error for the D_{1/2} value, and

4. Multiply the initial dilution of the serum by the averaged D_{1/2}.

This product is the final BP-HAI titer of the tested sample. For example, in one of the experiments with the post-vaccination serum of a donor, the D_{1/2} value was found to be 29.9. The regular HAI titer, and the corresponding pre-dilution for this serum was 1250. Therefore, the final BP-HAI titer was determined to be 29.9×1250=37427.

### Example 5

### Determination of BP-HAI Titers for H1N1 Solomon Islands Influenza Virus and a Sub-Pool of 15 Human Anti-Influenza Sera

VaxDesign possesses a pool of donor anti-influenza sera extracted from 18 donors total, the sera being drawn from each donor before and after vaccination (pre- and post-sera, respectively). Each serum was characterized by traditional HAI in January-March 2008. BP-HAI titers were determined for 15 sera out of the total of 36 in May, 2008.

Typical layouts for the BP-HAI experiments performed on the pools of human sera and MIMIC® samples (discussed in Example 8 below) are shown in FIG. 7, upper table. In these examples, experiments comparing BP-HAI and regular HAI, samples of four tested sera were pre-diluted according to their regular HAI titers determined previously, then sub-diluted 5, 15, and 45 times and placed in a 96-well format U-bottom plate, together with Virus, No Serum, and No Virus, No Serum samples, in duplicates, as shown in FIG. 7, upper table. In subsequent experiments, testing unknown sera or experimental fluids, no pre-dilutions were used, and the samples were serially diluted in the triple mode, as shown in FIG. 7, lower table.

The correlation between regular HAI and BP-HAI titers, plus the ensemble-average BP-HAI sub-titers for both high- and low-affinity models are shown in FIGS. 8A and 8B. The scattering plot of the BP-HAI data versus regular HAI demonstrated very good correlation between the two sets of the data (K_{corr}=0.993-0.998 using high- and low-affinity models for calculations). The general improvement of sensitivity using the BP-HAI assay, either as an ensemble-average D_{1/2} value, or as a slope coefficient of the BP-HAI/regular HAI scattering plot was estimated to be ˜19-23-fold, depending on the model used for the calculations. The low-affinity model generally demonstrated a better fit to the experimental data (compare the ensemble average standard deviations for the two models in the table in FIG. 8B). As a result, all the subsequent experimental data were processed using the low-affinity model curve “Dilution-BP events.”

### Example 6

### Determination of BP-HAI Titers for the Extended Pool of 33 Human Sera with H1N1 Solomon Islands Virus

BP-HAI titers for another 18 sera samples were determined in July 2008 using an independent preparation of human erythrocytes. The calculations of the BP-HAI titers were performed using the low-affinity model (Example 5, above), because it demonstrated a better fit and smaller dispersion for the D_{1/2} data. The newly acquired BP-HAI titration data were combined with those acquired in May 2008 (Example 5, above). The new datapoints of the scattered plot grouped tightly around the trend line found for the previous sub-pool, and all the BP-HAI datapoints demonstrated the same trend and the same level of correlation with the data of the traditional HAI titration (FIG. 9). The estimation of the sensitivity improvement obtained in this extended experiment was very similar to that obtained before, about 20-23-fold. Also, a repeated analysis of two sera from the sub-pool tested in May 2008 showed good reproducibility of the BP-HAI assay (the insert in FIG. 9).

### Example 7

### Determination of BP-HAI Titers for an Extended Pool of 34 Human Sera with the H3N2 Wisconsin Influenza Virus

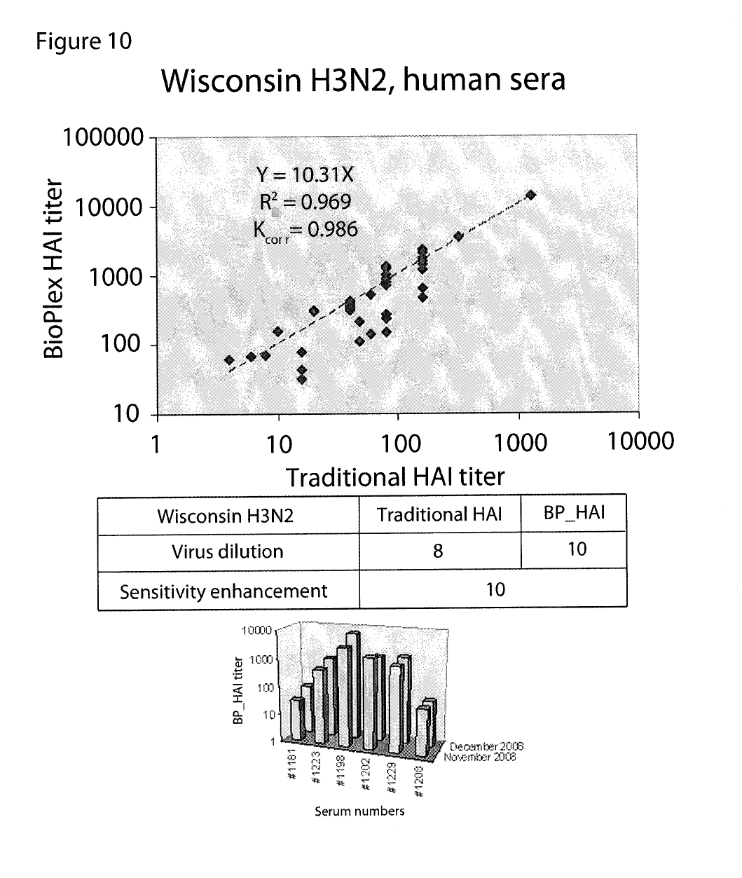

The H3N2 influenza virus type is a significantly weaker agglutinant than the H1N1 strain. Because of that, the conventional HAI assay with H3N2 Wisconsin used a dilution of the virus (BPL-inactivated CDC standard) of 1 to 8, compared with 1 to 40 with H1N1 Solomon Islands. It was found that H3N2 Wisconsin, when mixed with human group O ERC was also able to produce light-scattering objects detectable in the BioPlex® reader when a BP-HAI assay was performed. BP-HAI experiments performed using H3N2 Wisconsin in September-December 2008 showed good correlations with the regular HAI titers pre-determined for the sera with this virus earlier (FIG. 10). Those experiments used dilution of the H3N2 Wisconsin virus at 1 to 10, to compare with the 1 to 8 dilution used in the conventional HAI. The sensitivity of the BP-HAI assays with this virus was found to be improved approximately 10-fold versus the regular HAI (FIG. 10, bottom table).

### Example 8

### Determination of BP-HAI Titers for the Extended Pool of 32 MIMIC® Fluids with H1N1 Solomon Islands Virus

The MIMIC® (modular immune in vitro construct) system is an in vitro model of the human immune system devised at VaxDesign that enables researchers to test and down-select drugs, vaccines and chemical formulations before clinical trials, without using animal models or human volunteers. The MIMIC® system uses human blood cells under conditions similar to that in the human body, providing an in vitro testing system under more physiological conditions. When challenged with a pathogen, the MIMIC® is able to produce antibodies, similarly to an in vivo immune system. However, the concentrations of the antibodies produced are lower than in human or animal sera. In this regard, the new and more sensitive functional assay, the BP-HAI assay, is well suited for testing and analysis of MIMIC® samples.

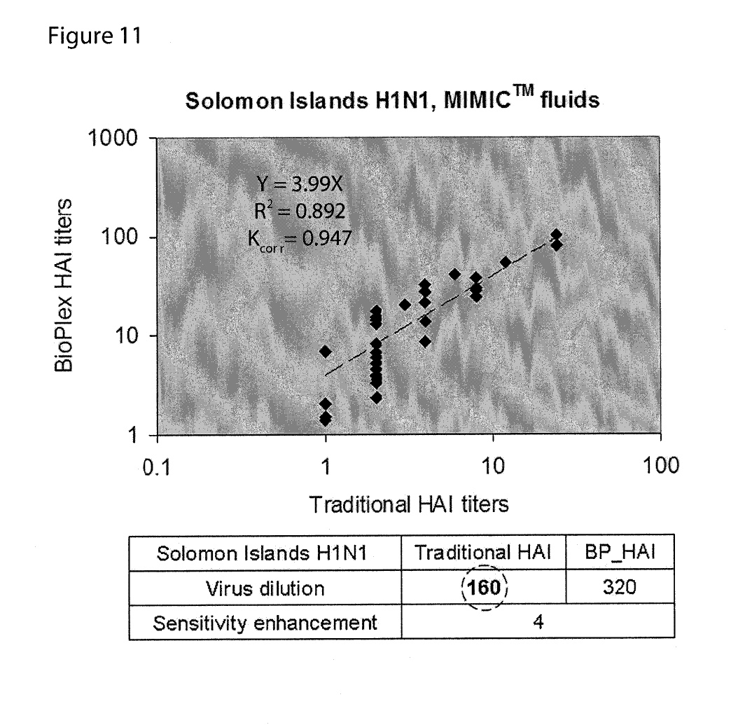

The MIMIC® system was ‘immunized’ in vitro with influenza vaccine over winter 2007/2008, and the resultant experimental fluids (supernatants) were tested for the presence of blocking anti-influenza antibodies, using traditional HAI assays and the BP-HAI assay of the present invention. Because of their insufficient sensitivity, however, the conventional HAI assays were performed with only the most efficient virus (i.e., with the H1N1 Solomon Islands virus, and at a dilution 4 times higher than with human sera: 1 to 160 for the MIMIC samples versus 1 to 40 for the human sera; FIG. 11, bottom table). The latter modification which actually deviated significantly from the commonly used HAI protocol, was able indeed to increase the sensitivity of the conventional HAI (the higher the dilution of the virus, the less antibody is necessary to block it), but only at the expense of its reliability.

The results of the regular HAI modified as indicated above were juxtaposed with the data of the BP-HAI acquired on the same MIMIC® samples. The scattered plot of the two data arrays (FIG. 11) showed acceptable correlation between the two methods, although compromised to some extent due to reduced reliability of the conventional HAI assays mentioned above. The improvement of sensitivity found in these experiments was also less significant than with human sera (˜4 vs. ˜20-23), and the reason for that was the increased dilution of the virus used in the conventional HAI assays. Indeed, assuming that the sensitivity of the HAI assay is roughly proportional to the dilution of the virus, then using the regular virus dilution, i.e., 1 to 40, instead of 1 to 160, in the HAI would lead to decrease of the sensitivity of the latter by ˜4 times. Against this level, the BP-HAI assay would show sensitivity improvement of ˜4×4=˜16, which is close to the sensitivity improvement found with human sera (˜20-23).

### Example 9

### Blocking of the Avian Influenza Virus by Polyclonal Antibodies and Human Sera Raised Against Seasonal Influenza A

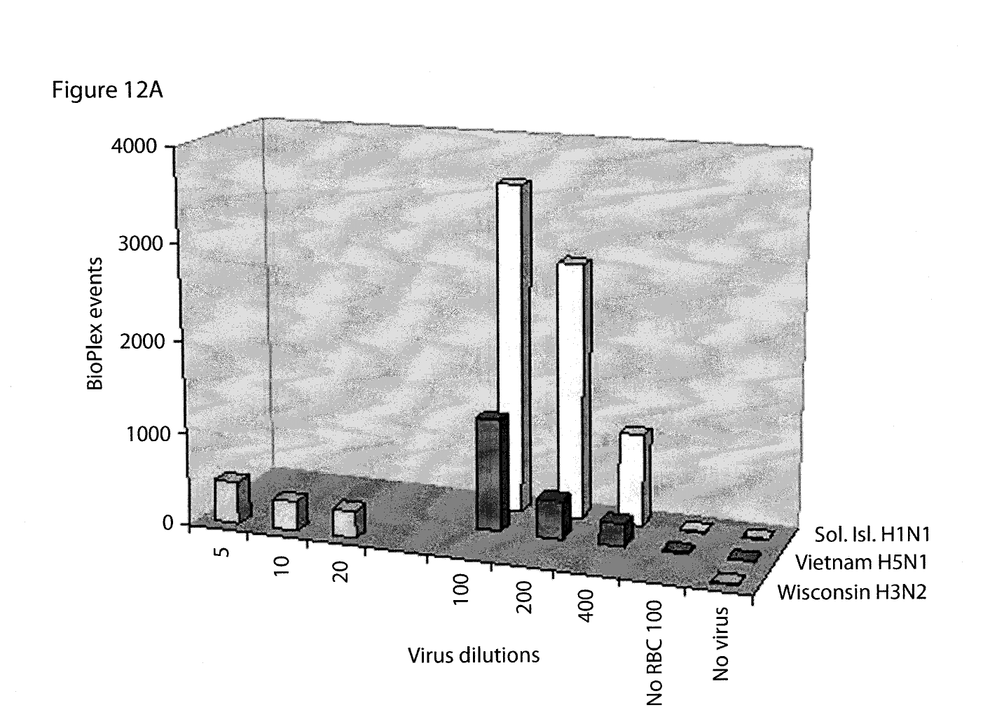

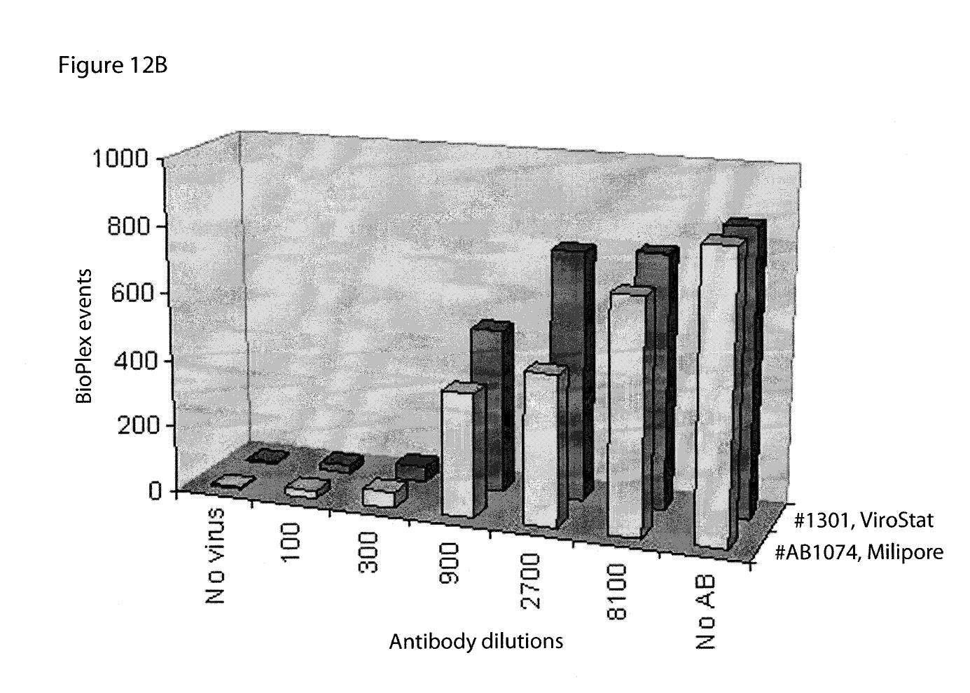

The virus of avian influenza, H5N1 Vietnam, has been demonstrated to have an agglutinating capacity somewhat lower than H1N1, but significantly higher than H3N2 (FIG. 12A). This makes H5N1 Vietnam a useful example for the BP-HAI assay of the present invention. It was found that polyclonal antibodies against seasonal influenza-A were able to block the H5N1 virus (FIG. 12B). Then, we tested sera from donors immunized against seasonal influenza regarding their capacity to block the avian influenza virus.

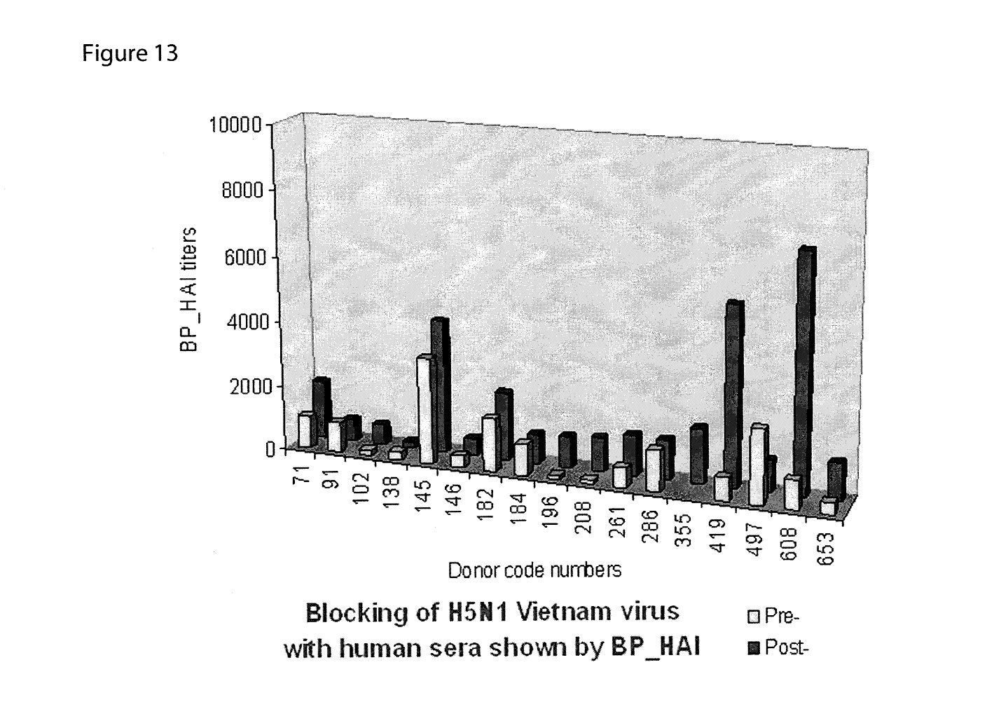

Screening of the pool of pre- and post-vaccination donor sera showed that although the average efficacy post-vaccination sera against the avian H5N1 Vietnam was low (the pool-averaged vaccination boost of the virus blocking was ˜2 for H5N1 to ˜15-17 for H1N1), the sera from some donors provided significant blocking effect (FIG. 13; donors #145, #419, #608). This data can help in determining epitopes important for blocking avian influenza and, therefore, in developing more effective vaccines that could prevent spread of potentially pandemic virus.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A hemagglutination assay, comprising:
(a) mixing erythrocytes and a sample of an agglutinating virus under conditions permitting agglutination of the erythrocytes, and
(b) detecting hemagglutination in the mixture of (a) by light scattering.

2. The hemagglutination assay of claim 1, wherein the erythrocytes are human erythrocytes.

3. The hemagglutination assay of claim 1, wherein the erythrocytes are human group O erythrocytes.

4. The hemagglutination assay of claim 1, wherein the virus is an influenza virus A or an influenza virus B.

5. The hemagglutination assay of claim 1, wherein the detection of light scattering in (b) is performed using a bead array reader.

6. The hemagglutination assay of claim 1, wherein the sensitivity of said assay is increased at least about 10-fold in comparison to a hemagglutination assay wherein the detection of hemagglutination is by the human eye.

7. The hemagglutination assay of claim 1, wherein the assay is repeated with at least one two-fold dilution of the sample of virus.

8. The hemagglutination assay of claim 1, wherein the sensitivity of said assay is increased at least about 10-fold in comparison to a hemagglutination assay wherein the detection of hemagglutination is by the human eye and wherein the detection of light scattering in (b) is performed using a bead array reader.

9. The hemagglutination assay of claim 1, wherein the detected amount of hemagglutination in the mixture of (a) is less than about 10 erythrocytes.

10. A hemagglutination inhibition assay, comprising:
(a) mixing a sample of an agglutinating virus and an antiserum under conditions permitting binding of an antibody in said antiserum to a hemagglutinin protein on the virus,
(b) mixing erythrocytes with the mixture of (a) under conditions permitting agglutination of the erythrocytes, and
(c) detecting hemagglutination in the mixture of (b) by light scattering.

11. The hemagglutination inhibition assay of claim 10, wherein the virus is an influenza virus A or an influenza virus B.

12. The hemagglutination inhibition assay of claim 10, wherein the antiserum is an antiserum that was raised against the virus.

13. The hemagglutination inhibition assay of claim 10, wherein the erythrocytes are human erythrocytes.

14. The hemagglutination inhibition assay of claim 10, wherein the erythrocytes are human group O erythrocytes.

15. The hemagglutination inhibition assay of claim 10, wherein the detection of light scattering in (c) is performed using a bead array reader.

16. The hemagglutination inhibition assay of claim 10, wherein the sensitivity of said assay is increased at least about 10-fold in comparison to a hemagglutination inhibition assay wherein the detection of hemagglutination is by the human eye.

17. The hemagglutination inhibition assay of claim 10, wherein the assay is repeated with at least one two-fold dilution of the sample of virus.

18. The hemagglutination inhibition assay of claim 10, wherein the assay is repeated with at least one two-fold dilution of the sample of antiserum.

19. The hemagglutination inhibition assay of claim 10, wherein the assay is repeated with at least one two-fold dilution of the sample of virus and at least one two-fold dilution of the sample of antiserum.

20. The hemagglutination inhibition assay of claim 10, wherein the sensitivity of said assay is increased at least about 10-fold in comparison to a hemagglutination inhibition assay wherein the detection of hemagglutination is by the human eye and wherein the detection of light scattering in (c) is performed using a bead array reader.

21. The hemagglutination inhibition assay of claim 10, wherein the detected amount of hemagglutination in the mixture of (b) is less than about 10 erythrocytes.

22. A method of detecting hemagglutination in a sample, comprising detecting hemagglutination in a sample by light scattering.

23. The method of claim 22, wherein the detection of light scattering is performed using a bead array reader.

24. The method of claim 22, wherein the sensitivity of the detection is increased at least about 10-fold in comparison to a hemagglutination assay or a hemagglutination inhibition assay wherein the detection of hemagglutination is by the human eye.

25. The method of claim 22, wherein the detected amount of hemagglutination in the sample is less than about 10 erythrocytes.
